(19) United States
(12) Patent Application Publication
(10) Pub. No.: US 2007/0093747 A1
(43) Pub. Date: Apr. 26, 2007

(54) Multiple dosage injection delivery apparatus

(76) Inventor: David Fredrick Smith, Santana de Pamalba (BR)

Correspondence Address: J. CHARLES DOUGHERTY, 200 WEST CAPITOL AVE, SUITE 2300, LITTLE ROCK, AR, 72201, US

(21) Appl. No.: 11/584,790

(22) Filed: Oct. 20, 2006

(30) Foreign Application Priority Data
Oct. 25, 2005 (BR) MU8502383-3

(51) Int. Cl. A61M 31/00

(52) U.S. Cl. 604/67; 604/131

(57) ABSTRACT

An inoculation system is disclosed in which a bird or other small animal is positioned over the inclined cover of a vaccination case being pressed against a sensor located in a plate mounted on the surface of the cover in order to activate the vaccination process. Once activated, an injection device is forced forward until it reaches the end of its course in its support. In this forward position, a needle attached to the injection device passes through the hole in the cover of the vaccinator case and penetrates the skin of the animal positioned over the hole, and two or more injectable materials are injected through the needle under the skin of the animal.

### CROSS-REFERENCE TO RELATED APPLICATIONS

This application claims the benefit of Brazilian patent application No. MU8502383-3, filed on Oct. 25, 2005, for which the inventor is David Frederick Smith. Such application is fully incorporated herein by reference.

### BACKGROUND OF THE INVENTION

The present invention relates to a device for the injection delivery of drugs, vaccines or other fluids into animals, with emphasis on birds. More particularly, the present invention allows the subcutaneous or intramuscular injection of two or more non-soluble drugs or vaccines through one needle into a bird.

Inoculation of one-day-old chicks or other small birds using automatic vaccine injection devices is known in the poultry industry. Automatic bird injection devices, including devices suitable for injecting small animals or birds such as one-day-old chicks are described, for example, in U.S. Pat. Nos. 5,312,353, 4,863,443, 4,758,227, 4,681,565, 4,276,879, 4,177,710, 4,108,176, 3,964,481, and 3,641,998. Such automated devices can allow one person to inoculate a multitude of birds with significant economic benefit through reduced labor costs.

These automatic injection devices generally provide a movable reciprocating carrier that supports a syringe with a single injection needle assembly connected to a fluid supply container. The syringe may be actuated relative to a support surface against which the chick is maintained by the operator. Once the needle attached to the syringe reaches its extended position penetrating into the tissue of the bird, the syringe plunger or other dose delivery means is actuated to deliver the required dose from the supply container to the recipient bird.

It may also be desirable to separately administer different drugs or vaccines. Most vaccines, antibiotics and probiotics can be mixed into the same vaccine delivery recipient because they are carried in a water-based diluent. But there are other vaccines that utilize diluents based in oil. These two types of vaccines, oil-based and water-based, cannot be mixed into the same vaccine delivery recipient. They would separate into two layers, thereby not allowing the two vaccines to be injected in the right proportions. Such combinations have to be either injected consecutively by two different needles or by two separate vaccinators into different localities in the recipient bird. The stress caused to the day-old chicks being vaccinated two or more times increases the mortality of the day-old chicks and thus increases the costs of applying these vaccines.

U.S. Pat. No. 4,758,227, for example, provides two injection needles configured to be simultaneously introduced into a grown bird's breast muscle tissue. This automatic injection system can inject two doses at the same time. However, the diminutive size of the intended recipient birds, such as one-day chicks, results in a limited area available for the automatic injectors to deliver the separate doses to the breast muscle tissue on opposite sides of the keel bone.

U.S. Pat. No. 6,789,467 provides two injection needles to simultaneously introduced vaccine via a subcutaneous route in the neck region. This injection system has serious limitations when utilized on day-old chicks. The neck region on a day-old chick is very small and very tender and any movement of the day-old chick while the needles are under the skin can provoke tears in the skin between the needles. Furthermore, with the two needles penetrating the tissue, there is an increase in the possibility of contaminants entering through these resulting wounds. There is also increased stress of having two needle penetrations in the small animal or bird.

Thus, there exists a need for an automatic inoculating system for small animals, especially for one-day-old chicks, that can effectively deliver two or more doses of non-soluble therapeutic fluids such as drugs or vaccines simultaneously via only one needle into a subcutaneous part of the body.

### SUMMARY OF THE INVENTION

The present invention provides a system for the precise injection delivery of at least two doses of non-soluble fluids into a small bird by the penetration of the recipient bird with only one injection needle. It is possible with the injection delivery system of the present invention to simultaneously inject two or mores vaccines, or other fluids that are not soluble, through the same injection needle at the same time without compromising the dosage of any of the liquids.

The present invention includes two or more dosage devices that individually control the exact quantity of each separate fluid to be transferred to the injector device via silicone or other tubing. Upon receiving the individual dosages of fluids, the injector device propels the injection needle forward to penetrate the skin of the recipient bird. Upon penetration, these two or more types of fluids are injected into the bird's body. The contact time of the fluids before being injected is so minimal that even non-soluble liquids that are also non-compatible in other ways can be injected together without adverse reactions. At the conclusion of the injection, the injector device retracts, withdrawing the needle from the skin of the recipient bird. At this time, the dosage devices have preferably already received new quantities of fluids that will be transferred to the injector device in preparation for the next injection.

In preferred embodiments of the present invention, the injection process is triggered by an activation mechanism being pressured through contact from the chick's body. This mechanism, which consists of a sensor or sensors attached to a plate that can be acrylic, is mounted in an assessable location, which can be on the surface of a stainless steel case housing the injector and dosage devices. When the neck, leg or wing of the bird is pressed against the one or more sensors, the sensor redirects the compressed air, compressed fluids or electricity to activate a micro-valve timer controller that allows compressed air, compressed fluids or electricity to enter the power cylinder that powers the injection device. When activated, the power cylinder forces the injection device forward to penetrate the skin of the bird. The injection device is seated in a support that allows it to move forward sufficiently so that a needle attached to the front of the injection device penetrates the flesh of the bird to a pre-determined depth. The power cylinder continues to exert pressure, pushing the plunger of the injection device forward and forcing the fluid contained in the chamber of the injection device to pass through the needle into the bird. When the plunger reaches its full course and the fluid has been injected, the micro-valve timer control shuts down the compressed air, compressed fluids or electricity and the injection device is pulled back by spring action, withdrawing the needle from the bird. As the plunger returns to its original position, the vacuum created by this process sucks the vaccine from the two or more dosage devices into the chamber of the injection device. Simultaneously, the plunging action of the dosage devices is forcing the vaccine out of its chambers.

The functioning of the dosage devices in the preferred embodiments of the present invention is very similar to that of the injection device although they remain in a fixed position. After delivering their dosage of fluids to the injector device, the compressed air, compressed fluids or electricity controlling their power cylinders is turned off by micro-valve timer control and the plungers are withdrawn from the chambers of the dosage devices via spring action. This action creates a vacuum that sucks vaccine into the chamber via silicone or other tubing attached to vaccine recipients outside the injection case. The dosage devices are immediately ready to deliver the liquid for the next injection. The dosage is determined by the size of the plunger and/or the size of the chamber.

These and other features, objects and advantages of the present invention, as described above with respect to certain preferred embodiments of the present invention, will become better understood from a consideration of the following detailed description of the preferred embodiments and appended claims, in conjunction with the drawings as described following:

### BRIEF DESCRIPTION OF THE SEVERAL VIEWS OF THE DRAWINGS

FIG. 1 is a partial cut-away view of a bird activating the sensor mechanism on the inclined lid of the vaccinator of the preferred embodiment of the present invention, with the injection needle penetrating and delivering the vaccine to the bird.

FIG. 2 is a partial cut-away view of an injection device that receives calibrated vaccine dosages from two dosage devices and injects them through a single needle into the bird being vaccinated according to a preferred embodiment of the present invention.

FIG. 3 is a partial cut-away view of a dosage device according to a preferred embodiment of the present invention, showing how the vaccine dosage is calibrated.

FIG. 4 is a partial cut-away view of the entire vaccine delivery system through one needle according to a preferred embodiment of the present invention.

### DETAILED DESCRIPTION OF THE PREFERRED EMBODIMENTS

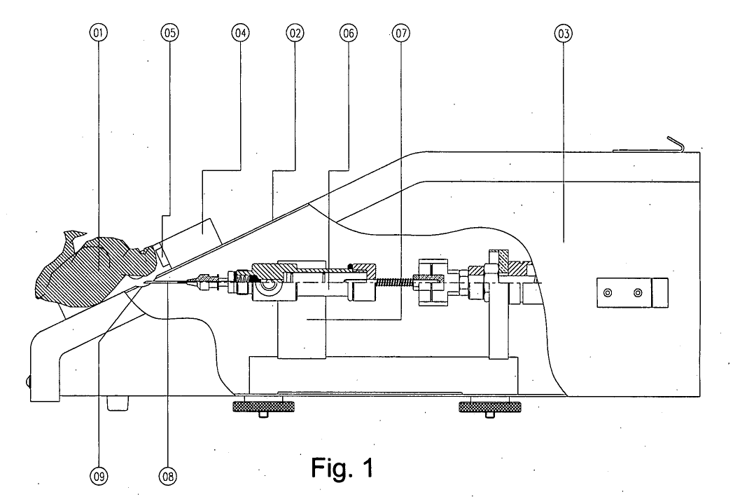

Reference will now be made in detail to the preferred embodiments of the invention, as illustrated in the accompany drawings, FIGS. 1-4. The drawings are to assist in the explanation of the invention, and do not limit the scope of the invention. It is apparent that skilled technicians could make modifications, combinations, variations, additions or deletions to the present invention without departing from the scope or spirit of the invention. It is intended that the present invention covers such modifications, combinations, additions, deletions and possible variations as within the scope of the appended claims.

The preferred embodiment of the present invention is used to simultaneously deliver multiple dosages of vaccines or other injectable but insoluble substances to small animals such as chicks. Many injectable materials cannot be mixed into the same delivery system because they are not soluble, such as oil-based and water-based vaccines. The injection delivery system of this invention allows for the accurate dosage in one injection of incompatible materials.

FIG. 1 represents a bird 1 being placed on the slanted lid 2 of the vaccinator 3 and being pushed against a plate 4 mounted on the surface of the slanted lid 2 that contains a sensor 5 to activate the injection process. Upon activation, the injection device 6 is forced forward until it reaches the extent of its course in the injection device body support 7. In its forward movement the single needle 8 attached to the injection device 6 passes though a hole 9 in the lid 2 of the vaccinator 3 and penetrates the skin of the bird or small animal positioned over the needle exit hole 9 and two or more injectable materials are injected through the single needle 8 under the skin of the bird or small animal.

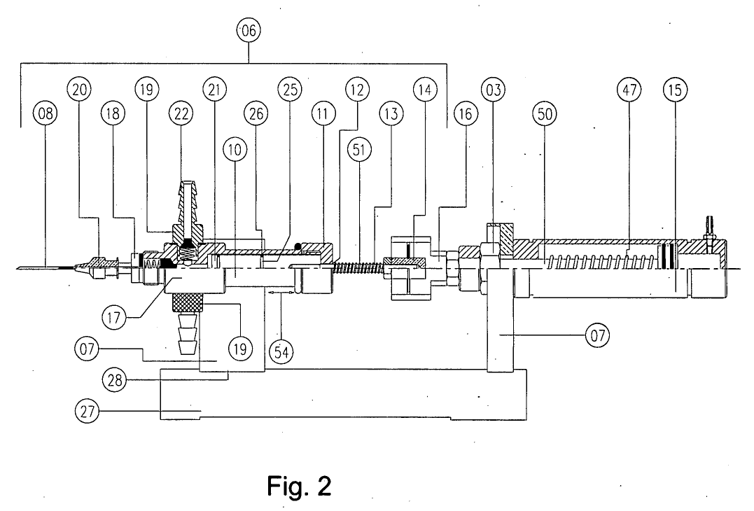

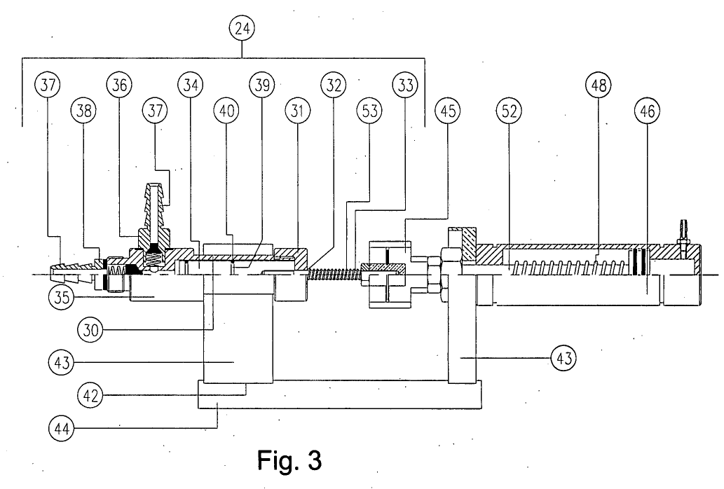

FIG. 2 illustrates a cutaway of the injection device 6 utilizing one needle 8 capable of delivering two or more injectable substances simultaneously. The injection device 6 consists of a cylindrical chamber 10 threaded on one end with an end cap 11 screwed onto the threads, the cap which has a hole 12. The plunger 21 is inserted into the cylindrical chamber 10 and its control rod 13 passes through the hole 12 in the end cap 11, so that the plunger control rod end piece 14 is connected to the plunger 50 of the power cylinder 15 by a snap-in quick connector 16. The control rod 13 has a spring 51 whose function is to return the plunger 21 of the injection device to the rear of the cylindrical chamber after injection. On the front end of the chamber 10 is located a head piece 17. The head piece 17 contains a forward flow control apparatus 18 to which is attached a conical nipple 20 over which the injection needle 8 is fitted. This forward flow control apparatus 18 is configured to open its outlet during the forward movement of the injection device plunger 21 in the direction of the needle 8 and to close its inlet during the backward movement of the plunger 21. The head piece 17 also contains two or more lateral flow control apparatuses 19 with ribbed nipples 22 over which silicone tubing 23 is affixed for connection to the dosage device 24, as shown in FIG. 3, and which are configured to close their outlets within the chamber 10 during the forward movement of the plunger 21 in the direction of the needle 8 and to open their inlets during the backward movement of the plunger 21 to allow the liquid from the dosage device 24, as shown in FIG. 3, to enter the chamber 10. The plunger 21 has two milled grooves 25 with O-rings 26 placed in them. When the plunger 21 is inserted into the chamber 10, these O-rings create a tight seal against the inner wall of the chamber 10. The plunger 21 is adapted for reciprocal movement within the chamber 10 such that fluid from the dosage device 24, as shown in FIG. 3, is drawn through silicone tubing 23 into the chamber 10 through the inlet port of the lateral flow control apparatus 19 by the backward motion of the plunger 21, and such that fluid is expelled into the injection needle 8 from the chamber 10 through the forward flow control apparatus 18 by a forward motion of the plunger 21.

The injection device 6 is held in position on its base plate 27 by a body support 7 with a slot 28, allowing the injection device 6 to be inserted and removed with relative ease. When the power cylinder 15 is activated, the air pushes forward the power cylinder plunger 50 that is connected by the snap-in quick connector 16 to the plunger control rod 13 of the injection device 6, pushing the injection device 6 forward. The body support 7 allows this forward movement of the injection device 6 until the end cap 11 at the end of the injection device 6 reaches the encounter point 54 of the support 7 which breaks the forward movement. Therefore, the power cylinder plunger 50 attached to the injection device 6 moves the entire injection device 6 forward until the end cap 11 reaches the support 7 and then the power cylinder plunger 50 continues to move only the plunger 21 forward, forcing the injectable material out of the chamber 10. The limited forward movement of the injection device 6 is the movement that causes the injection needle 8 to penetrate the bird or small animal.

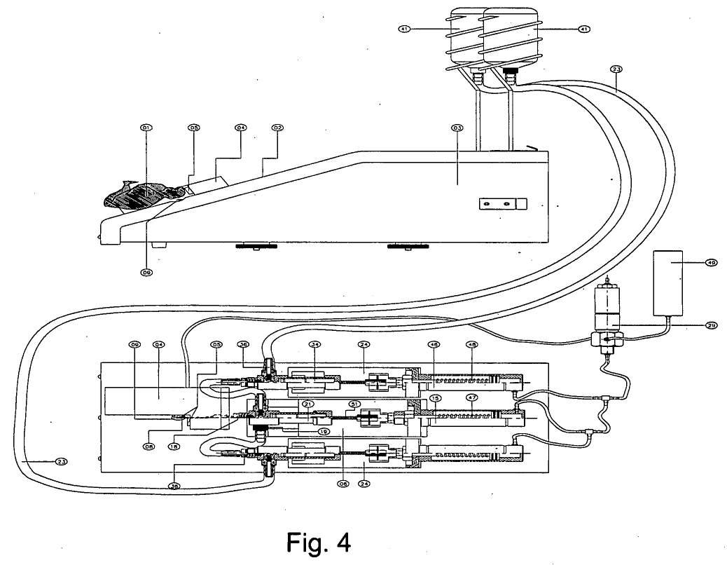

A power cylinder plunger 50 controls the forward and backward movements of the injection device 6 in its body support 7 and also the forward and backward movements of the injection device plunger 21. When a small animal or bird is pushed against the sensor or sensors 5, as shown in FIG. 1, the flux of the compressed air, compressed fluids or electricity is altered to activate the micro-valve timer controller 29, as shown in FIG. 4, which directs the compressed air, compressed fluids or electricity to the power cylinder 15, which will force the injection device 6 forward to inject the bird or other small animal. Once the injection device 6 has reached its maximum thrust, determined by the encounter point 54 of the end cap 11 with the body support 7, the power cylinder plunger 50 will continue to force the injection device plunger 21 forward, forcing the injectable material out of the chamber 10 and through the needle 8 mounted on the head 17 of the injection device 6. The injectable material flows through the single needle 8 into the small animal or bird in contact with the sensor or sensors 5, as shown in FIG. 1, of the plate 4.

After the plunger 21 of the injection device 6 reaches the end of its course and the injectable material has been forced from the chamber 10 of the injection device 6 through the needle 8 and into the animal, the micro-valve timer controller 29, as shown in FIG. 4, deactivates the power cylinder 15 that is returned to its original position via spring action 47, pulling the plunger 21 of the injection device 6 to its starting position in the rear of the chamber 10, removing the needle from the bird. When the power cylinder removes the pressure holding the injection device plunger forward, the plunger spring 51 pulls the plunger back to its original position at the rear of the cylindrical chamber 10. The movement of the plunger to the rear of the chamber creates a vacuum because the O-rings 26 in contact with the chamber walls 10 form a tight seal. Consequently, the next dosage of injectable material is drawn into the chamber 10 of the injection device 6 by force of the vacuum, aided also by the forward movement of the dosage device plungers 24, as shown in FIG. 3, pushing the injectable material out of the dosage devices.

The injection device 6 is positioned on its base plate 27 with the needle 8 oriented toward the front of the vaccinator 3, as shown in FIG. 1, and aligned so that when activated the injection device 6 will slide forward in its support 7 and as it moves forward the needle 8 will pass through a hole 9 in the vaccinator box lid 2, as shown in FIG. 1. As it passes through the hole 9, as shown in FIG. 1, it penetrates the skin of the small animal or bird.

FIG. 3 illustrates the dosage device 24 that controls the passage of injectable material between its original recipient 41, as shown in FIG. 4, and the injection device 6, and determines the quantity of this material injected into the bird. Using an individual dosage device 24 for each fluid to be added to the total injected fluid means that the volume of each liquid is controlled and the dosage from each dosage device 24 can be a different quantity. Therefore, liquids that are non-soluble can be vaccinated together without affecting their volumes.

The dosage device 24 consists of a cylindrical chamber 30 threaded on one end with an end cap 31 screwed onto the threads, the cap which has a hole 32 through which passes the control rod 33 of the plunger 34 which is inserted in the chamber 30. On the other end of the chamber 30 is located a head piece 35. The head piece 35 contains two lateral flow control apparatuses 36 and 38 to which are attached ribbed nipples 37 over which silicone tubing 23, as shown in FIG. 4, is affixed. The inlet lateral flow control apparatus 36, whose silicone tubing 23, as shown in FIG. 4, is connected to a supply container of liquid 41, as shown in FIG. 4, is configured to open its inlet during the backward movement of the plunger 34 to draw the liquid into the dosage device 24, and to close its outlet when the plunger 34 is moved forward. The outlet lateral flow control apparatus 38 with ribbed nipple over which silicone tubing 23 connected it to the injection device 6 is configured to close its inlet within the chamber 30 during the backward movement of the plunger 34 and to open its outlet during the forward movement of the plunger 34.

The plunger 34 has two milled grooves 39 with O-rings 40 placed in them. When the plunger 34 is inserted into the chamber 30, these O-rings 40 create a tight seal against the inner wall of the chamber 30. The plunger 34 is adapted for reciprocal movement within the chamber 30 such that fluid from the injectable liquid container 41, as shown in FIG. 4, is drawn through silicone tubing 23 into the chamber 30 through ribbed nipple 37 on the inlet lateral flow control apparatus 36 and when moved forward, that fluid is expelled into the injection device 6, as shown in FIG. 2, through silicone tubing connected to the ribbed nipple 37 on the outlet lateral flow control apparatus 38. The dosage device 24 is calibrated by the size of plunger 34 and/or chamber 30 being utilized.

Chamber 30 of the dosage device 24 is mounted in a slot 42 on a support 43 attached to a base plate 44 and the control rod 33 of the dosage device 24 is secured within a quick-connect support 45 attached to a plunger 46 of the power cylinder 15 mounted on the same base plate 44. The quick-connect on the plunger 34 and the slotted support 43 allows the dosage device 24 to be inserted and removed with ease, but holds the dosage device 24 secure in this position.

The dosage device 24 delivers its injectable liquid via the silicone or other tubing that is connected to the ribbed nipple on the outlet port on the head 38 of the dosage device 24. The other end of this tubing is connected to one of the ribbed nipples 37 on the inlet port of the injection device 6, as shown in FIG. 2. The calibrated dosage passes from the dosage device 24 to the injection device 6, as shown in FIG. 2, which is now ready for the next injection. As soon as the injectable material is passed to the injection device 6, as shown in FIG. 2, the micro-valve timer controller 29, as shown in FIG. 4, cuts the compressed air flow to the plunger 46 of each dosage device 24. The plunger spring 48 pulls the power cylinder plunger 52 back until the plunger reaches to end of the power cylinder and the dosage device plunger spring 53 pulls the dosage device plunger 34 backwards until the plunger reaches the end of the chamber 30.

FIG. 4 illustrates the complete unit of the dosage and injection devices for the delivery of two injectable materials with one injection needle 8. The system begins with the injectable material containers 41 that can be made of any sterilizable material such as glass, plastic bags, etc. Silicone or other tubes 23 are connected between two containers and their respective inlet lateral flow control apparatuses 36 with the tubing to the dosage device 24 placed on the ribbed nipple attached to the inlet apparatus. Other silicone or other tubes are connected between the ribbed nipples mounted on the outlet lateral flow control apparatuses 38 of the dosage device 24 and the ribbed nipples on the lateral flow control apparatus 19 of the injection device 6. Once the silicone or other tubing is in place the vaccinator 3 is ready to delivery two injectable materials through a single injection needle 8 mounted on the forward flow control apparatus 18 on the injection device 6.

Because of the inclination of the lid 2 relative to the position of the injection device 6, the needle 8 does not penetrate the bird perpendicularly. The needle 8 penetrates the skin almost parallel to the bird's neck.

To begin the process, a small animal or bird 1 is placed on the inclined lid 2 of the vaccinator 3 and slid against the sensor plate 4, as shown in FIG. 1. The pressed sensors 5 redirects the compressed air, compressed fluids or electricity to activate a micro-valve timer controller 29 that allows compressed air, compressed fluids or electricity 49 to enter the power cylinders 15 and 46 of the injection device 6 and the dosage devices 24. The power cylinder 15 advances, pushing the plunger 21 forward, forcing the injectable material through the needle. The micro-valve timer controller 29 turns off the flow of compressed air and the power cylinder retracts by spring action 47, taking with it the injection device 6 that removes the needle from the bird and, finally, the plunger spring 51 retracts the plunger to the rear of its chamber. Simultaneously, the micro-valve timer controller 29 permits the compressed air to enter the power cylinder of the dosage device 24. The power cylinder 46 advances, pushing the dosage device forward, forcing the injectable material to pass through the outlet lateral flow control apparatuses 38 and enter the injection device 6. Working together, the retraction of the plunger of the injection device 21 produces a vacuum that draws the liquid from the dosage device 24 into the injection device 6. The cut in air flow to the dosage device plunger by the micro-valve timer controller 29 provokes a retraction of the power cylinder caused by its spring 48, bringing the dosage device plunger 34 to the rear of its chamber 30, creating a vacuum that draws the injectable liquid into its recipient 41 within the cylindrical chamber 30, ready for a bird to activate the sensor again for the next dosage to be administered.

The present invention has been described with reference to certain preferred and alternative embodiments that are intended to be exemplary only and not limiting to the full scope of the present invention as set forth in the appended claims.

I claim:

1. An animal inoculation apparatus, comprising: a cover comprising a hole; a sensor mounted adjacent to the cover; an injection device comprising a needle and operable to extend in response to a signal from the sensor whereby the needle passes through the hole in the cover to inoculate the bird positioned over the hole; and a plurality of dosage devices in communication with the injection device and operable to pass a plurality of fluids through the injection device and the needle and into the animal simultaneously.

2. The apparatus of claim 1, further comprising a plurality of lateral inlet flow control apparatuses operable to receive the fluids.

3. The apparatus of claim 2, further comprising a plurality of inlet flow control apparatuses each for receiving a different fluid from a different dosage device.

4. The apparatus of claim 2, including only one needle, wherein the apparatus is operable to inject a composite of the plurality of fluids, and wherein the composite is formed of fluids wherein at least one of the fluids is non-soluble with respect to at least one of the other fluids.

5. The apparatus of claim 2, wherein the dosage devices are operable to simultaneously deliver the fluids in individually pre-determined dosages through the needle into the animal.

6. The apparatus of claim 2, wherein the dosage devices are operable to simultaneously deliver the fluids in different quantities through the needle into the animal.

7. A vaccination apparatus, comprising: a slotted support; and a dosage device firmly fixed in the slotted support.

8. The apparatus of claim 7, wherein the dosage device is operable to deliver a precise dosage of a liquid in the range of 0.01 ml to 2 ml.

9. The apparatus of claim 7, further comprising a plunger and a dosage chamber, and wherein the apparatus is operable to adjust a dosage of a liquid by altering at least one of the size of the plunger and the size of the dosage chamber.

10. The apparatus of claim 7, further comprising a plunger in communication with the dosage device, and wherein the forward movement of the plunger acts to deliver a liquid from the dosage device.

11. The apparatus of claim 7, further comprising a plurality of lateral flow control apparatuses and a plunger, and wherein the lateral flow control apparatuses are operable to prevent liquid from being transferred to the dosage device when the plunger is in a backward movement.

12. The apparatus of claim 7, further comprising a liquid supply chamber; a plunger; and a plurality of lateral flow control apparatuses operable to prevent a liquid from being transferred from the apparatus to the supply chamber when the plunger is in a forward movement.
